(19) United States
(12) Patent Application Publication
(10) Pub. No.: US 2004/0138688 A1
(43) Pub. Date: Jul. 15, 2004

(54) Lancet system including test strips and cassettes for drawing and sampling bodily material

(76) Inventor: Jean Pierre Giraud, Paris (FR)

Correspondence Address: GREENBERG TRAURIG, LLP, 885 3RD AVENUE, NEW YORK, NY, 10022, US

(21) Appl. No.: 10/683,692

(22) Filed: Oct. 9, 2003

Related U.S. Application Data
(60) Provisional application No. 60/417,201, filed on Oct. 9, 2002.

(51) Int. Cl. A61B 17/32

(52) U.S. Cl. 606/181

(57) ABSTRACT

A lancet suited for use in drawing blood or other bodily fluid, wherein the lancet is provided with a region where a chemical reagent resides, the chemical reagent including one or more agents that react with at least one constituent of the bodily fluid to ascertain information about the bodily fluid from which it was drawn, the lancet has a body and a head that is provided with a relatively sharp tip for penetrating the skin of the person, the tip is provided with at least one channel or groove in fluid communication with the area where the chemical reagent is positioned, the channel or groove transports the fluid from the tip to the area where the chemical reagent is positioned, the channels extend between the tip of the lancet and the recess, an electroconductive ink is positioned on the lancet wherein the ink is patterned into electrical contacts, part of which extend into the area where the chemical reagent is deposited and a part of which extend to a location away from the area where the chemical reagent is deposited, the chemical reagent positioned on the lancet undergoes a reaction with the bodily fluid that creates an electrical potential between the two ends of the electrical contacts.

### CROSS-REFERENCE TO RELATED APPLICATIONS

This application claims the benefit under 35 U.S.C. 119(e) of U.S. Provisional Application Serial No. 60/417,201, filed Oct. 9, 2002.

### FIELD OF THE INVENTION

The present invention is directed to lancets, cassettes, diagnostic devices, and/or related devices employed in the drawing of a sample of a bodily material, and/or the storage and/or analysis of the material.

### SUMMARY OF THE INVENTION

In one embodiment, the present invention is a lancet that is suited for use in drawing blood or other bodily fluid from a human being or other animal. (For the sake of simplicity, only “person” shall be referred to in this paper). The lancet is provided with a region where a chemical reagent resides, the chemical reagent including one or more agents that react with at least one constituent of the bodily fluid to ascertain information about the bodily fluid and/or body from which it was drawn. For example, in some instances, the bodily fluid may be blood, and the chemical reagent may be one that, when it reacts with the blood, undergoes a chemical reaction providing useful information. In one embodiment, the information can be used to determine the blood sugar level of the body from which it was drawn. Such information would be useful to a diabetic, who, in reliance upon the information concerning the level of blood sugar obtained with use of the lancet, would know the insulin dosage to administer himself.

In another embodiment, the lancet of the present invention has a body and a head that is provided with a relatively sharp tip for penetrating the skin of the person. The tip is provided with at least one channel or groove in fluid communication with the area where the chemical reagent is positioned. The channel or groove transports the fluid from the tip to the area where the chemical reagent is positioned. In one specific embodiment, a recess is provided on the body of the lancet. The recess houses the area where the chemical reagent is positioned. The channels extend between the tip of the lancet and the recess. In yet a further embodiment, an electroconductive ink is positioned on the lancet. The ink is patterned into electrical contacts, part of which extend into the area where the chemical reagent is deposited (which may be the aforedescribed recess), and a part of which extend to a location away from the area where the chemical reagent is deposited. In this embodiment, the chemical reagent positioned on the lancet undergoes a reaction with the bodily fluid that creates an electrical potential between the two ends of the electrical contacts, which can be measured by a diagnostic device, which can be used to learn useful information, such as the presence and/or level of a constituent in a bodily fluid. To give yet another example, a user may sample a fluid to determine if she is pregnant.

In one embodiment, the lancet has a pencil-like shape, with the head resembling the conically-shaped pencil point and the body resembling a cylindrical barrel. However, the lancet may be formed into suitable shapes and sizes other than the pencil-shaped lancet shown in the figures.

As an illustrative example, the lancet may be employed to sample bodily fluids such as urine, blood, saliva or other fluid. For example, in the case of glucose testing, a blood sample is drawn using an invasive method (i.e., with a Lancet). In one example, the skin is penetrated with a lancet, creating a blood spot (up to 10 μl) and wetting a test strip with the blood.

In a further embodiment, the lancet of the present invention can be integrated into a diagnostic test kit in which the lancet is mechanically driven out of a protective recess when a sample is taken. For example, the lancet can be driven out of the recess to penetrate a person's skin, draw a sample of a fluid, such as blood, which by capillary action is transported through the grooves to the area where the reagent is positioned. The lancet is returned to within the protective recess. The chemical reaction occurs, generating an electrical potential, which is measured by the diagnostic test kit, which then converts the value of the electrical potential to useful information, such as a blood sugar level. This information is displayed in a window on the device.

In one embodiment, the lancet of the present invention may be produced by injection molding a plastic material. The lancets of the present invention are intended to be disposable and, in one embodiment, a one-time use product.

While the present invention has apparent use in processes that will provide diagnostic information in the time immediately following the drawing of a sample, it also may be used as a sampling device employed in clinical laboratory testing. There are many quantitative tests that, for one reason or another, are not performed by the person who drew the sample. In these instances, the lancet is provided with a storage reservoir for storing a sample of fluid that has been withdrawn. Testing may be performed on the sample immediately after the sample is drawn or the sample may be transported to a laboratory for testing at a later time. Where the lancet is to be used in this way, the area where the chemical reagent is positioned may also include a preservative, to retain the sample's viability in the period of time prior to analysis.

In one embodiment, the lancets of the present invention can be manufactured by an in-mold labeling process wherein the parts are molded, and, also within the mold, labeled with an electrically conductive ink that forms the pattern of electrical contacts on the lancet. In another embodiment, the electrical contacts may also be applied to the lancet via pad printing electrically conductive ink.

In yet another embodiment, the present invention is a cassette for retaining a plurality of lancets, positioning them for penetration into the user's body, in order to draw a sample, mobilizing them in the sample collection process, retaining them for sample analysis, and then retaining the lancets after they have been used. In one embodiment, the lancets are linked by a ribbon, and the lancet-ribbon assembly is placed with the cassette, which then draws new lancets into the mobilization position and disposes of them in a compartment when after they have been used.

Yet another embodiment of the present invention relates to a test strip for use in analyzing a sample drawn from a person, as described above. The test strip has a substrate, a through hole where a chemical reagent can be deposited and cured, electrical contacts that extend from where the reagent is positioned to another area on the strip. This arrangement allows for the determination of an electrical potential, which can be used to determine useful information about the presence and/or level of a constituent in the bodily material. In a specific embodiment, the strip also contains, at an end thereof, a wicking region comprised of grooves containing a non-compatible ink (with respect to the plastic material that forms the substrate). The wicking region transports bodily fluid from the end of the strip to the region where the chemical reagent is deposited. In one example, the strip has an overlayer of a carrier material. In a further embodiment, the strip can be formed by an in-mold labeling process in which the electrically conductive inks and the non-compatible ink are patterned on the carrier, which is then labeled on the strip as it is formed in the mold.

### BRIEF DESCRIPTION OF THE DRAWINGS

FIG. 1 is a perspective view of a lancet of the present invention;

FIG. 2 is a cross sectional view of a lancet of the present invention along line A-A of FIG. 1;

FIG. 3 is a cross sectional view of a lancet of the present invention along line B-B of FIG. 1;

FIG. 4 is a schematic of a mold in which lancets of the present invention are created by an in mold labeling method;

FIG. 5 is a schematic of a mold in which lancets of the present invention are created by a pad printing method;

FIG. 6 is a perspective view depicting an alternative arrangement to lancets of the embodiment shown in FIG. 1;

FIG. 7 is a perspective view showing an arrangement for linking a plurality of lancets;

FIG. 8 is a perspective view showing yet another arrangement for linking a plurality of lancets;

FIG. 9 is a perspective view showing yet another arrangement for linking a plurality of lancets;

FIG. 10 is a perspective view showing a cassette for storing lancets, delivering them for penetration into the person's body, and analyzing the sample taken from the person;

FIG. 11 is a cross sectional view of the cassette;

FIG. 12 is a cross sectional view of the cassette drive mechanism, loaded with lancets;

FIG. 13 is a perspective view showing an aspect of the cassette;

FIG. 14 is a perspective view showing an aspect of the cassette;

FIG. 15 is a perspective view of a test strip of the present invention;

FIG. 16 is a cross sectional view of the test strip of FIG. 15, taken along line A-A; and

FIG. 17 is a cross sectional view of the test strip of FIG. 15, taken along line B-B.

### DETAILED DESCRIPTION OF THE EMBODIMENTS

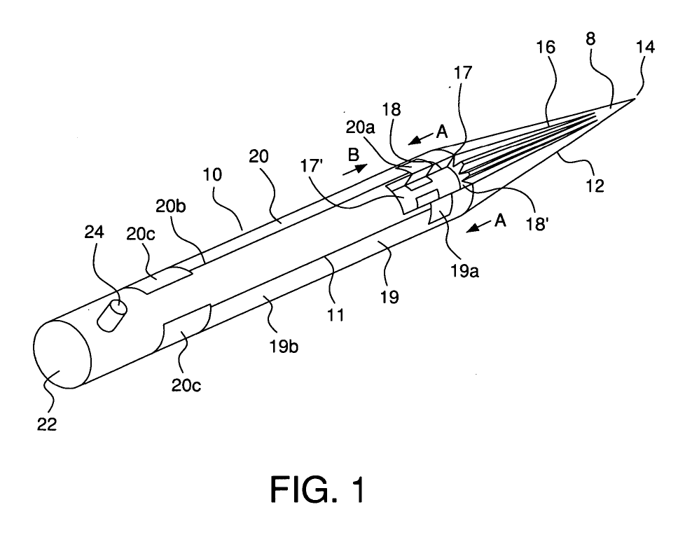

FIG. 1 shows one embodiment of a lancet 10 of the present invention. As shown, the lancet of the present invention has a body 11 and a head 12 that is provided with a relatively sharp tip 14 for penetrating the skin of the person. The tip 14 is provided with a collecting area 8 for collecting the fluid sample. The collecting area is positioned between the tip 14 and the grooves 16 in fluid communication with the area 17 where the chemical reagent (not shown) is positioned. The grooves 16 transport the fluid from the tip 14 of head 12 to the area 17 where the chemical reagent is positioned. As shown in FIG. 1, in one example, the area 17, where a chemical reagent is stored, is a recess 17′ provided on the body of the lancet. Recess 17′ is defined by sidewalls 18. The grooves 16 are formed in one of the sidewalls 18′. See FIGS. 1 and 3. In another example, the area for chemical reagent is positioned co-planar with the remainder of the body.

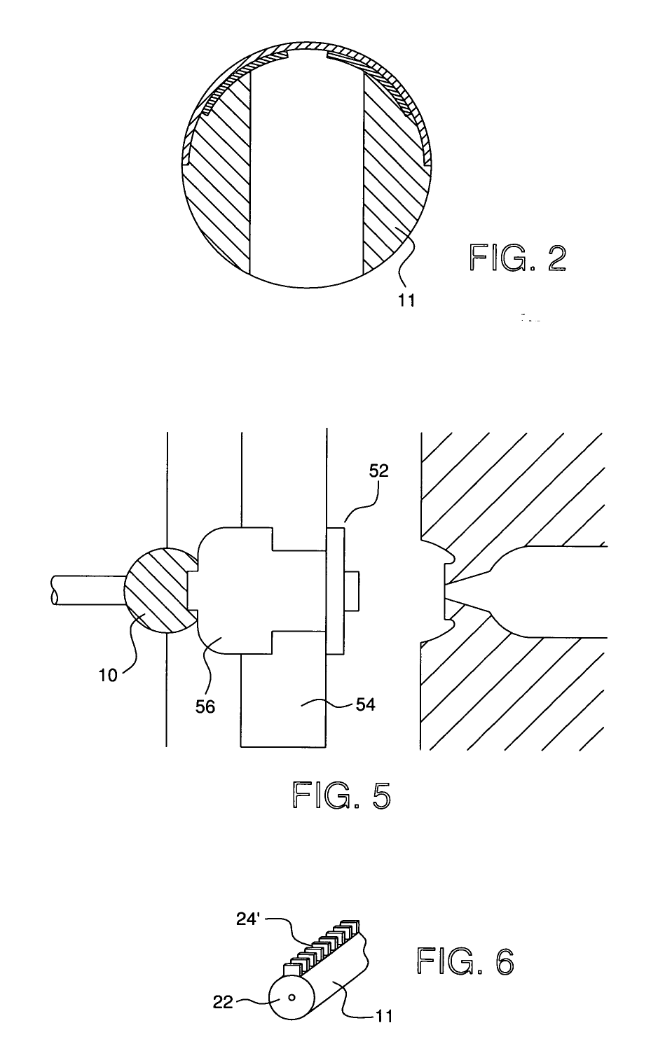

Grooves 16 extend along the surface of the head 12, between the tip 14 of the lancet and the recess 17′. In embodiments where the chemical reaction between the sample taken from the person and the chemical reagent yields information that can be analyzed electrically, an electroconductive ink is positioned on the lancet. As shown in FIG. 1, in one example, the ink is patterned into a pair of electrical contacts 19 and 20. Electrical contacts 19 and 20 are provided with proximal laterally extending portions 19a and 20a, which, as shown in FIG. 1, extend into the recess 17′, where the chemical reagent is deposited. See FIGS. 1 and 3. Electrical contacts 19 and 20 are further provided with distal laterally extending portions 19c and 20c, positioned towards the distal end 22 of the body. See FIGS. 2 and 3. Digits 19b and 20b of the contacts 19 and 20 provide the electrical contact between the proximal laterally extending portions 19a and 20a of contacts and the distal laterally extending portions 19c and 20c of contacts. Digits 19b and 20b extend in a direction that is substantially coaxial with the body.

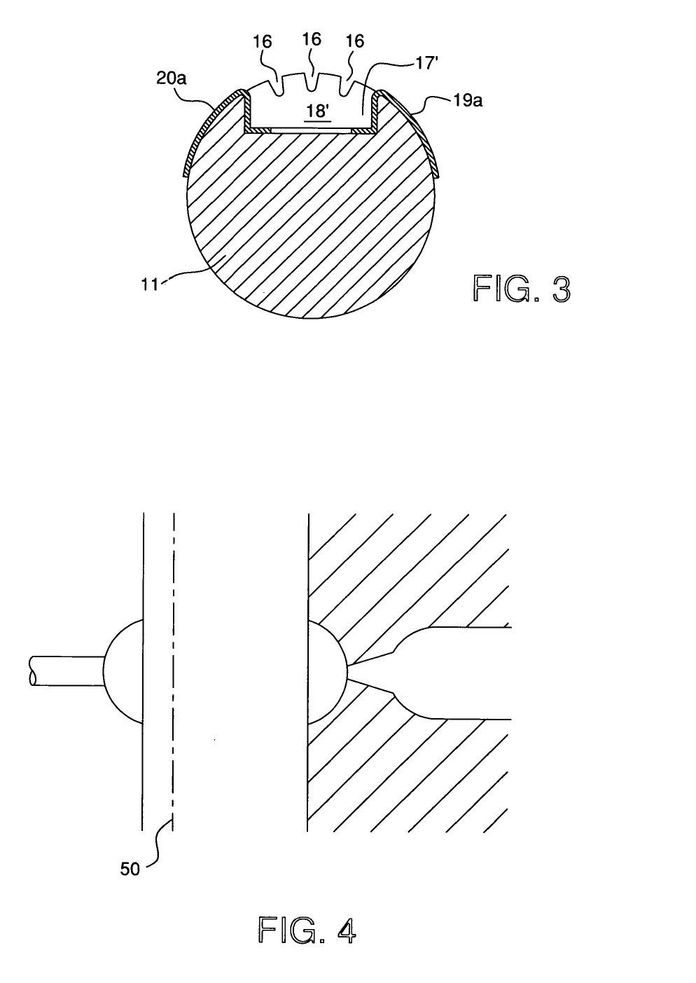

In one embodiment, the distal end of the pin is further provided with a transport pin 24 extending substantially perpendicular to the axis of the body. The transport pin 24 interfaces with a driver device that drives the lancet into a penetrating, sample obtaining position, and back to a rest position. In an alternative embodiment, shown in FIG. 6, a rack and pinion arrangement 24′ is provided near the distal end 22 of the body. The rack and pinion arrangement mates with a driving structure provided in a diagnostic device or cassette, such as a wheel having a gear face on its perimeter. The interface between the geared wheel and the rack and pinion arrangement provides structure which can drive the lancet as described in this paragraph and thus, other comparable drive means can also be employed.

Since the lancet is considered a Class 1 medical device by the U.S. Food and Drug Administration “(FDA”), the materials used to construct it should be approved for Class 1 use. These materials include: Acetal (POM), polypropylene, polyethylene and performance plastics.

In one embodiment, the lancet can be made in a single injection molding process, in which electrical contacts 19 and 20 are applied to the lancet in the mold, by an in-mold labeling process, such as depicted in FIG. 4. The in-mold label 50, comprised of a substrate layer and a conductive ink layer patterned on the substrate in the pattern in which the conductive ink is to be applied to the lancet, is placed in the mold and the thermoplastic is injected into the mold. See FIG. 4. The injection pressure of the plastic forces the label against the cavity wall opposite the point where the thermoplastic is injected.

In another embodiment, shown in FIG. 5, a pad printing process, such as one employing a pad-printing robot, is used to apply a conductive ink in the preselected pattern of the electrical contacts 19 and 20. For example, the robot arm 54 moves a printing pad 56 in and out of contact with the lancet 10. Here, the ink is applied to the already molded part. In both embodiments (in-mold or pad-print) the conductive material is applied in-mold.

The lancets can be molded on a relatively small vertical molding machine provided with high cavitation so that, for example, 50 or more lancets can be made at a time. In one embodiment, the molding occurs in an environmentally controlled room, that is one where temperature and humidity are controlled, and contaminants are maintained to below acceptable levels. In one specific example, the manufacturing process may be comprised of:

1. Indexing the in mold labels for all mold cavities;

2. Over mold lancet and label;

3. Open the mold

4. Robotically dispense the active chemical reagent to the appropriate location;

5. Cure the reagent;

6. Eject the parts from the mold;

7. Load the lancets in a cassette or other distribution device; and

8. Close the cassette.

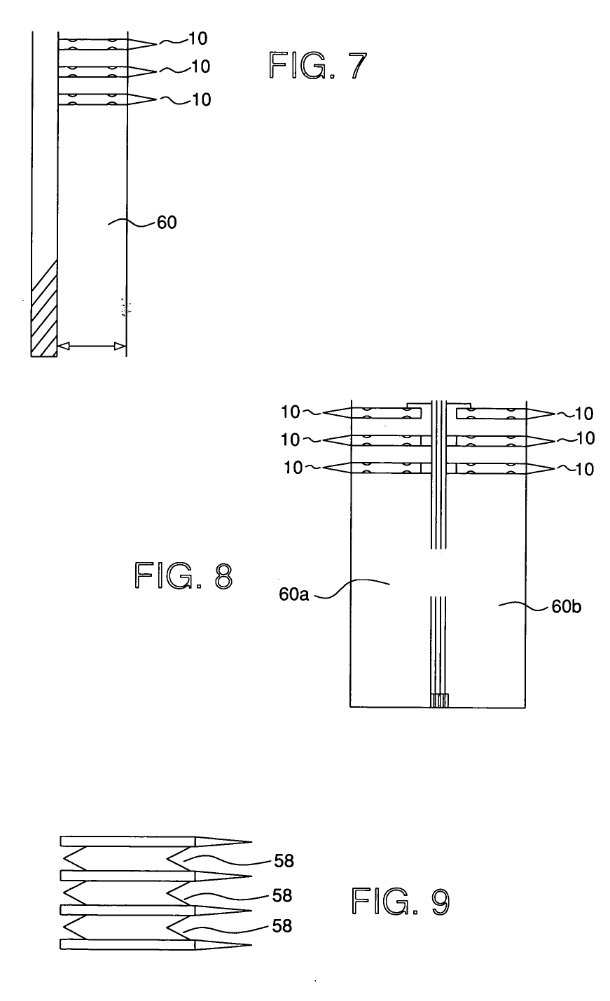

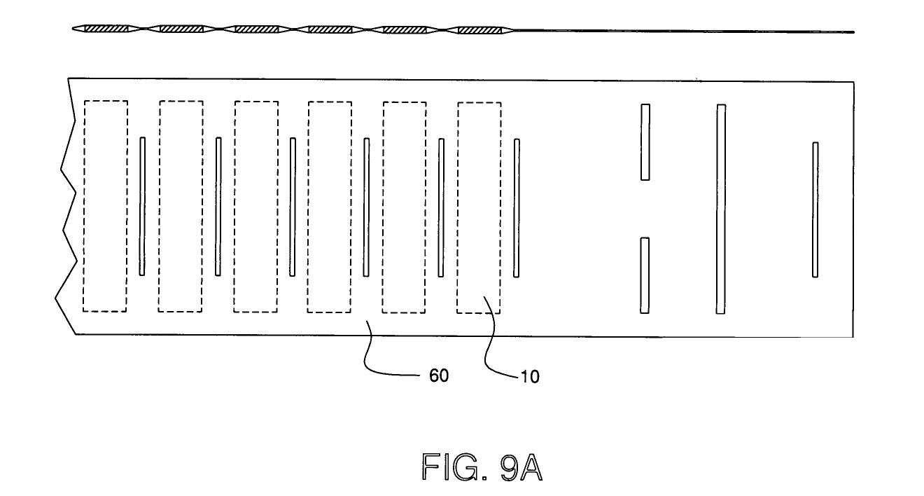

FIGS. 7, 8, and 9, depict another embodiment showing a manner of unitizing a number of lancets in order to retain, package and load them into a dispensing device. In FIGS. 7 and 8, the lancets are mounted to a continuous ribbon or sheet 60. The film is advanced in the injection-molding machine, and the lancets are molded directly on the film. The lancets are attached so that at least the heads 12 extend over the ribbon. In the embodiment of FIG. 8, the lancets are arranged on both sides of the ribbon, which is cut in the middle, to provide two sheets of lancets. In a further embodiment, a conductive material can be printed on the ribbon in a separate operation, or pad printed onto it in the mold. The ribbon, with the conductive material, can be used to test the conductive performance of the disposable units, providing an on-line quality testing system during manufacturing. FIG. 9 shows an alternative embodiment for unitizing lancets, in which collapsible accordion-like appendages 58 link individual lancets. In this case, the lancets are pad printed.

In one embodiment, 50 to 100 lancets are linked together as described herein. In yet another embodiment, an identifying tag, such as a bar code, is applied to each individual lancet, and/or ribbon. The code can be used to identify product lot number, and in the case where sample analysis occurs at a location different from where the sample is taken (such as a diagnostic laboratory), it can be used to supply information about who drew the sample, when it was drawn, and from whom the sample was drawn, to name three possible pieces of information. Also, where the sample is to be sent off to an offsite lab, the area where the chemical reagent is positioned may incorporate preservatives (i.e., EDTA, Heparin, etc) to insure the sample remains viable.

The Cassette

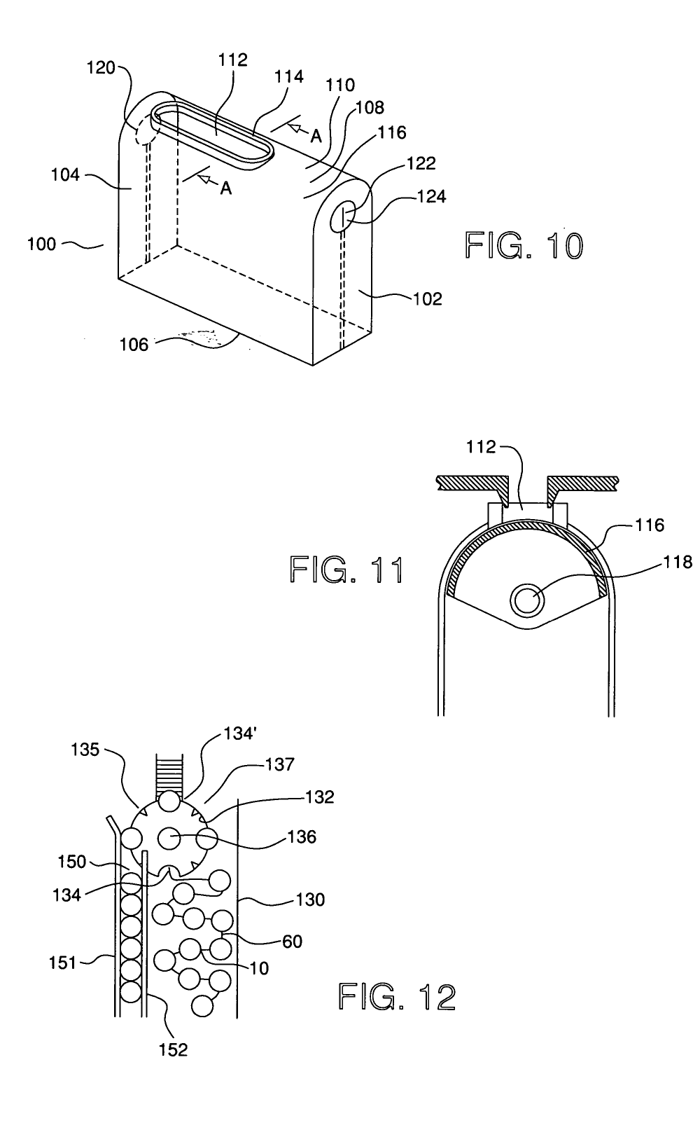

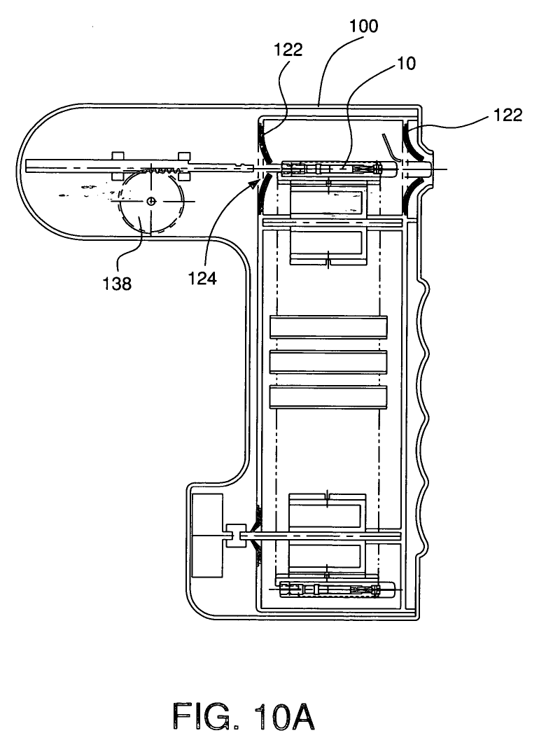

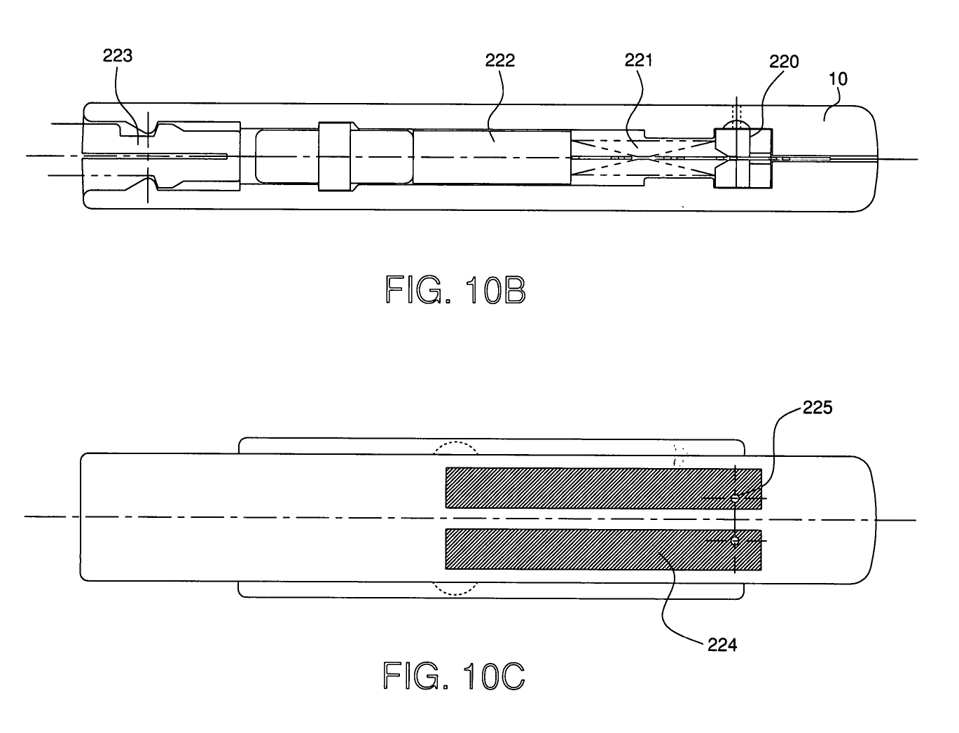

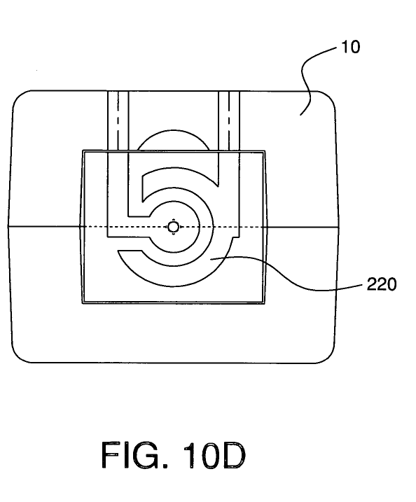

In a further embodiment, a cassette holds multiple lancets, typically 50 and 100 units. One embodiment of the cassette 100 is shown in FIG. 10. The cassette 100 is provided with first and second arched shaped ends 102, 104, and sidewalls 106, 108, and arcuate top surface 110, each of which are joined to the ends 102, 104. It should be understood that other shapes can be adapted for the cassette.

The cassette has an opening 112 positioned on the arcuate top surface 110. Here, the opening 112 is shown as having straight sides and rounded edges, though other shapes are possible. In one specific embodiment, the edges of the opening are lined with an elastomeric material 114, which extends into the interior of the cassette. In another example, on the interior of the cassette, in the area of the arcuate top surface 110, a window 116 is mounted to the twin axis 118. The window is rotatable between an open position and a closed position by a rack and pinion arrangement. The window closes when the cassette nears its final position in the diagnostic kit, which is provided with a rack which engages axis 118 for opening the window 116 through the membrane 120 provided on second arched shaped end 104.

In yet another embodiment, first arched shaped end 102 is provided with a membrane 122 having a slit 124 through which the head of the lancet passes when it is activated in order to obtain a sample from a person.

The cassette is provided with an interior housing 130 into which the lancets are loaded. As shown in an embodiment of FIG. 12, the lancets 10 are attached to the ribbon 60, and the ribbon and lancets are loaded into the interior housing in a serpentine arrangement. The lancets and ribbon fill the interior housing and are loaded into the distribution wheel 132, which indexes the lancets in preparation for their activation, and drawing of a sample. As shown, the distribution wheel 132 has four slots 134, though additional slots may be provided. The axis 136 of the distribution wheel is mounted to the twin axis 117 provided on the cassette 100. The activation position is the uppermost slot 134′ and the lancet in this slot engages with a driver, such as gear wheel 138, which moves the lancet out of the cassette and diagnostic kit, in order to take a sample. The distribution wheel 134 is provided with a series of indents 135, which receive a cutting blade 137 which cuts the ribbon of the lancet that is positioned in the activation slot 134′.

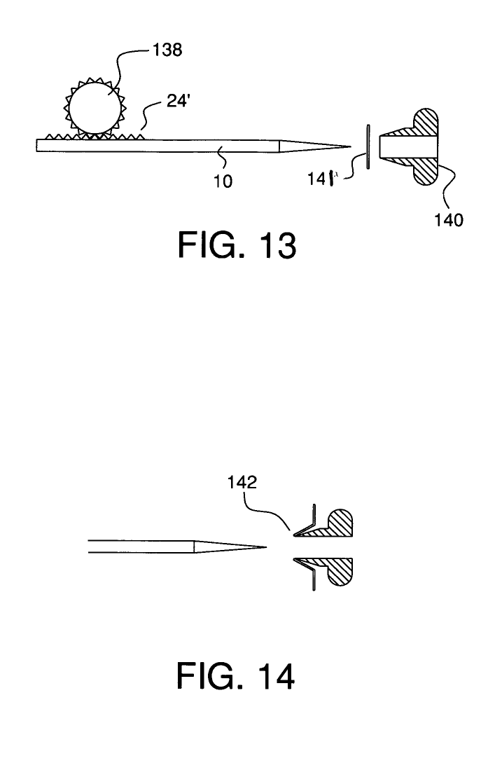

To draw a sample, shown in one embodiment as illustrated in FIG. 12, the user activates the device by issuing the appropriate command on the diagnostic kit, such as by pressing a button. In another embodiment, the membrane 124 is pressure sensitive and acts as switch, so that when the pusher mechanism is pressed against the user's skin, at the location where a sample is to be drawn, the stepper motor (or other suitable device) is activated, driving the lancet out of the cassette. In any event, when the stepping motor is activated, it drives a device such as the gear wheel 138 shown in FIGS. 12 and 13, which is engaged with the rack and pinion arrangement 24′ of FIG. 6. The stepping motor turns the gear wheel in a direction that drives the lancet towards the membrane 122, and the slit 124. The skin pusher 140 is moved back into the cassette, and contacts a membrane that is provided with an opening 142. The pusher opens the membrane 140, and the head of the lancet passes through the opening. The lancet penetrates the skin of the user, drawing a sample of fluid, such as blood. The blood enters the grooves on the lancet through the collecting zone, and is transferred by capillary action to the area where the chemical reagent is located. After a short period, the sample is collected. This process may take about a fraction of a second, which may be about 0.1 second. The lancet is retracted, by reversing the direction of the stepping motor. Optionally, the device emits an audible sound that notifies the user that the sampling process is completed. Once sample is collected, and the lancet retracted, the skin pusher returns to its original position. The membrane closes and re-establishes a moisture-tight seal in the meter.

In another embodiment, a protective film covers the surface of the skin pusher to prevent carryover of sample from test to test or collection to collection. The tip penetrates the protective film during a sampling cycle. The protective film is indexed to a new position for the next sampling cycle.

In the embodiment relating to a chemical assay, when the sample enters the area where the chemical reagent is positioned, a chemical reaction between the two takes place. The reaction may yield qualitative information, such as a color change, which the user analyzes by visual observation, or which is analyzed by the device. Alternatively, where the lancets are provided with the previously described electrical contacts, the chemical reaction may create an electrical potential between the proximal laterally extending portions 19a and 20a of contacts and the distal laterally extending portions 19c and 20c of contacts. The lancet is placed in electrical contact with electronics of the diagnostic kit. The potential is measured by the diagnostic device, and converted to useful information. For example, where the sample is blood, the potential can yield information on the user's blood sugar. This information is displayed on a window on the device.

In one embodiment, when the user takes the next test, a motor rotates the distribution wheel 132. For example, as shown in FIG. 12, the wheel is rotated in a counterclockwise direction. The rotation of the wheel (1) deposits the used lancet into a receptacle 150 within the interior housing, defined by walls 151 and 152, and (2) pulls another lancet into the lowermost slot 134″ on the wheel. These walls help to avoid cross-contamination between the used and unused lancets. During this process, the ribbon of the lancet in the activation slot 134′ is severed, as described above.

After all lancets in a cassette are used, the cassette is removed from the kit and disposed. A new cassette is inserted into the kit. When the cassette is removed, the window is automatically closed. Where the lancets are used for glucose testing, the meter is typically used by a single patient. In the case of a sampling device, the device may be used on multiple patients.

As an example of an application, the device described herein can be used at the patient's bedside in hospitals to collect sample. The reservoir may contain compounds that preserve the sample for future use. Once all samples are collected, each sample (contained within the reservoirs of each lancet) is dispensed into a sample preparation system. Sample preparation consists of, but is not limited to (1) Dilution; (2) Buffer addition; (3) DNA Amplification.

The device of the present invention can be used to collect samples including, but not limited, to the following situations: (1) Pediatric sample collection and testing; (2) Forensic testing; (3) General Hospital Use; (4) Clinical Laboratory collection sites; (5) Physician Offices (sample collection in a box).

Each person's skin quality varies in surface tension, thickness, overall toughness and vascularization. Moreover skin quality varies from site to site on a user's body. Alternative sampling is widely performed with blood glucose testing. In one embodiment, the present invention adjusts the force and depth of the lancet plunge based on the skin quality and the level of vascularization. The sampling function is adapted for each individual user (and for multiple sampling sites on a single user). Thus, it is believed that in one example, the optimization of the lancet plunge is determined by measuring the resistance on the linear motor that directs the lancet into the skin. The speed and force of the motor is powered and controlled by the test kit device. The resistance on the motor is monitored by the test kit system during lancet skin penetration. There is a defined ‘typical’ range of resistance. A computer algorithm adjusts the motor speed and power during subsequent penetrations (in the same body location) until the resistance in the typical range. The optimal motor speed and power is stored in a memory chip on the sampling device.

In a further embodiment, the plunge depth and dwell time in the user's body are determined by monitoring the rate sample collection in the lancet. In a specific embodiment, sample collection is monitored by incorporating a sensor near the lancet reservoir or tip. For example, if the lancet is made of polycarbonate plastic, the lancet is transparent. In another example, an infrared light source (i.e., diode) can be position along the distal end of the lancet, and shines down the center of the lancet during sample collection. A sensor positioned at a point near the collection reservoir or tip can monitor changes in light transmission—due to sample collecting in the reservoir or at the tip.

U.S. Pat. Nos. 5,494,562, 5,202,261, and 6,192,891 disclose the construction and operation of electrochemical-type and their employment in diagnostic assays. They are incorporated herein by reference.

In another embodiment, a visible light-emitting diode can be positioned along the distal end of the lancet to identify the location of skin penetration to the user.

In another embodiment, a desiccant plastic can line at least a portion of the inside walls of the cassette, to create a moisture free environment within the cassette. For example, a desiccant plastic disclosed in U.S. Pat. No. 6,174,852 (hereby incorporated by reference) may be employed here.

In one embodiment, the lancet has a diameter of about 1.5-2.0 mm and a length of about 13.0-15.0 mm long. The lancet is tapered along the pointed end—the tip has a series of grooves about 50-200 μm in depth extend along the tapered portion from the tip to the sample reservoir.

Test Strips

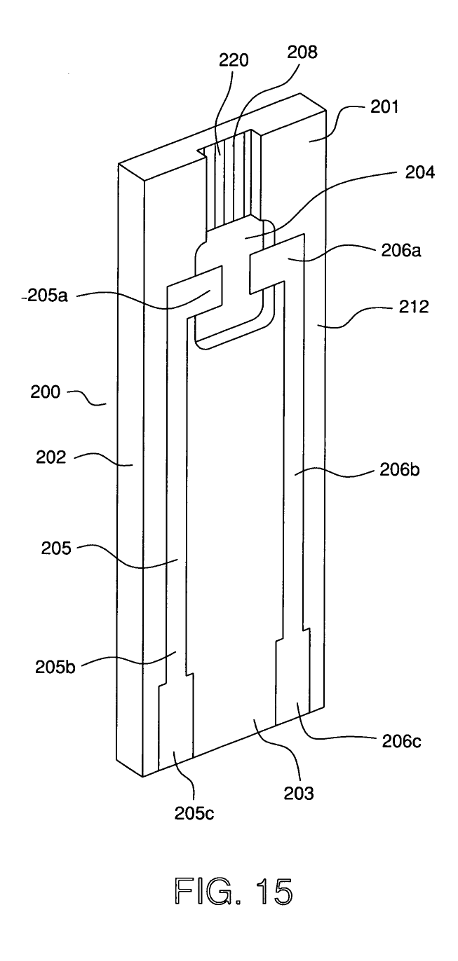

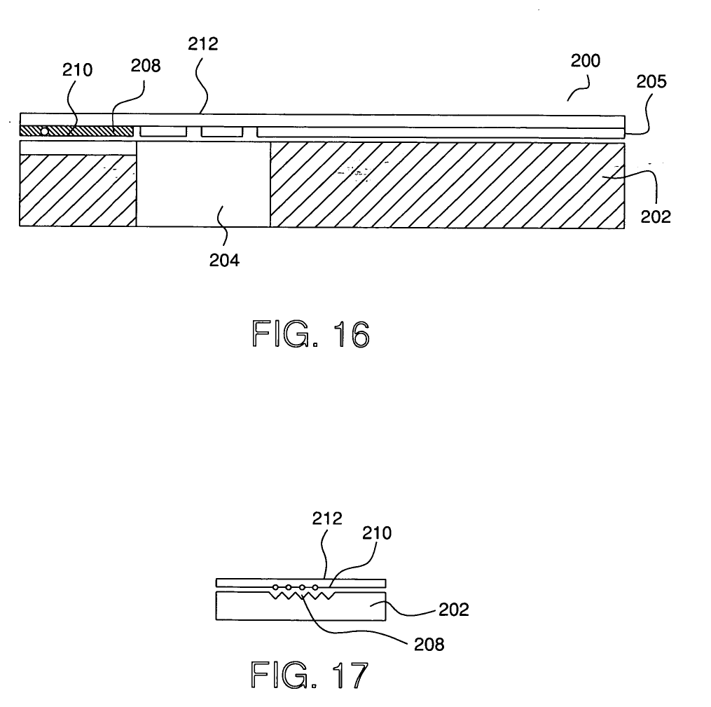

In another embodiment, the present invention is a test strip that employs a minimal number of layers. The strip 200 of the present invention is shown in one embodiment, illustrated in FIGS. 15-17. The strip is comprised of a substrate 202 having a proximal end 201 and a distal end 203. The substrate is provided with a through hole 204, which, during manufacturing, is filled with a chemical reagent selected to react with the sample drawn onto the substrate. The reagent is deposited in the hole and cured, as described above.

Electrical contacts 205 and 206 are positioned on the substrate 202. The electrical contacts have proximal laterally extending portions 205a and 206a, which extend into the opening 204. The laterally extending portions are in contact with digits 205b and 206b of the contacts 205 and 206. The digits extend in the direction of the length dimension of the strip. The digits are in contact with terminals 205c and 206c, of the contacts 205 and 206. The terminals are located proximate to the distal end 203 of the strip.

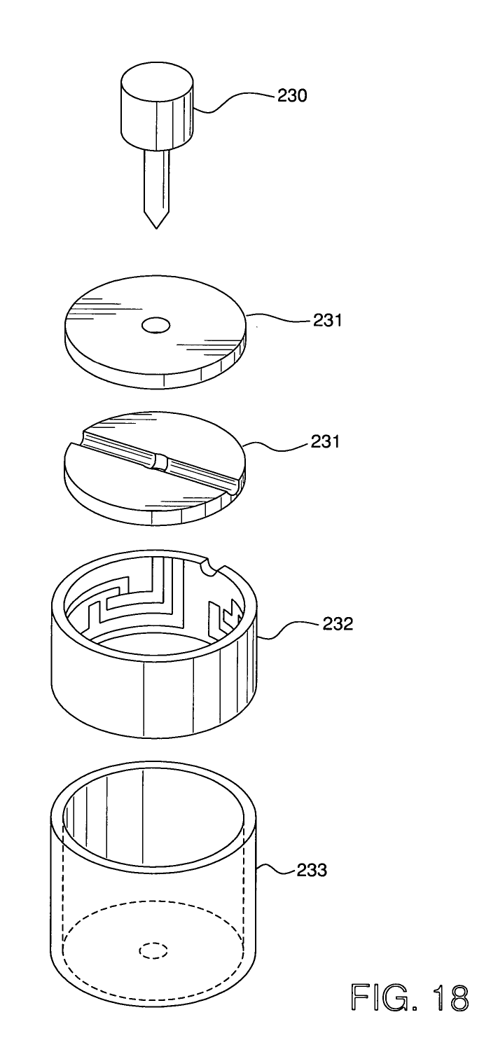

At the proximal end 201 of the strip 202, a pitted region 208 is positioned between the proximal end 201 and the through hole 204. A layer of an in-mold label 212 is deposited over the substrate. The in-mold label 212 layer is comprised of a carrier layer and two discreet coatings of ink. The first ink coating is a conductive ink, which is positioned on the carrier in the pattern of the electrically conductive inks, extending from the through hole to the distal end of the strip. The second ink layer is a non-compatible, non-adhesive ink, positioned on the carrier to be deposited in the region 208 between the proximal end of the strip and the through hole 204. By non-compatible, the ink is not compatible with the plastic material that is molded into the substrate. Accordingly, during the practice of the in-mold labeling method employed in producing the strips of the present embodiment, the ink does not bond with the substrate forming plastic, and instead forms its own discreet region positioned between the substrate layer and the IML carrier layer. Thus, during formation of the strip, the ink forms a series of grooves positioned between the substrate and the IML carrier. The grooves extend from the distal end to the through hole, as best shown in FIGS. 15 and 17. The ink is present in the finished strip. When a sample is drawn from a person, by placing the distal end of the strip in the fluid to be sampled, the ink acts as a wick, facilitating the transport of the fluid from the distal end, through the grooves, to the through hole.

In another embodiment, the strips are contained in a canister having a “new strip” drum and a “used strip” drum, with the two drums being interconnected by a channel. This canister resembles the canister for storing and moving 110 mm film. Strips are located in the new strip drum. The strips are laminated on to a film with tractor feed holes along each side. The tractor feed is driven by a sprocket system in the meter. The film is indexed—use strips collect in the old strip drum. The channel may be provided with a window or windows through which a sample can be deposited on the strip, and through which the strip may be analyzed. When all of the strips are used, the lancet is discarded and a replaced with a new lancet with the appropriate units (e.g. 50). A desiccant plastic can be used to line at least a portion inside of the lancet drum. For example, a desiccant plastic disclosed in U.S. Pat. No. 6,174,852 may be employed here.

What follows is a listing of tests and test systems listed in part 862 of Title 21 of the Code of Federal Regulations. The embodiments disclosed herein can be employed in conducting at least some of the tests enumerated below:

Part 862—Clinical Chemistry and Clinical Toxicology Devices

Subpart A—General Provisions

§862.1—Scope.

§862.2—Regulation of calibrators.

§862.3—Effective dates of requirement for premarket approval.

§862.9—Limitations of exemptions from section 510(k) of the Federal Food, Drug, and Cosmetic Act (the act).

Subpart B—Clinical Chemistry Test Systems

§862.1020—Acid phosphatase (total or prostatic) test system.

§862.1025—Adrenocorticotropic hormone (ACTH) test system.

§862.1030—Alanine amino transferase (ALT/SGPT) test system.

§862.1035—Albumin test system.

§862.1040—Aldolase test system.

§862.1045—Aldosterone test system.

§862.1050—Alkaline phosphatase or isoenzymes test system.

§862.1060—Delta-aminolevulinic acid test system.

§862.1065—Ammonia test system.

§862.1070—Amylase test system.

§862.1075—Androstenedione test system.

§862.1080—Androsterone test system.

§862.1085—Angiotensin I and renin test system.

§862.1090—Angiotensin converting enzyme (A.C.E.) test system.

§862.1095—Ascorbic acid test system.

§862.1100—Aspartate amino transferase (AST/SGOT) test system.

§862.1110—Bilirubin (total or direct) test system.

§862.1113—Bilirubin (total and unbound) in the neonate test system.

§862.1115—Urinary bilirubin and its conjugates (nonquantitative) test system.

§862.1117—B-type natriuretic peptide test system.

§862.1118—Biotinidase test system.

§862.1120—Blood gases (PCO2, PO2) and blood pH test system.

§862.1130—Blood volume test system.

§862.1135—C-peptides of proinsulin test system.

§862.1140—Calcitonin test system.

§862.1145—Calcium test system.

§862.1150—Calibrator.

§862.1155—Human chorionic gonadotropin (HCG) test system.

§862.1160—Bicarbonate/carbon dioxide test system.

§862.1165—Catecholamines (total) test system.

§862.1170—Chloride test system.

§862.1175—Cholesterol (total) test system.

§862.1177—Cholylglycine test system.

§862.1180—Chymotrypsin test system.

§862.1185—Compound S (11-deoxycortisol) test system.

§862.1187—Conjugated sulfolithocholic acid (SLCG) test system.

§862.1190—Copper test system.

§862.1195—Corticoids test system.

§862.1200—Corticosterone test system.

§862.1205—Cortisol (hydrocortisone and hydroxycorticosterone) test system.

§862.1210—Creatine test system.

§862.1215—Creatine phosphokinase/creatine kinase or isoenzymes test system.

§862.1225—Creatinine test system.

§862.1230—Cyclic AMP test system.

§862.1240—Cystine test system.

§862.1245—Dehydroepiandrosterone (free and sulfate) test system.

§862.1250—Desoxycorticosterone test system.

§862.1255-2,3-Diphosphoglyceric acid test system.

§862.1260—Estradiol test system.

§862.1265—Estriol test system.

§862.1270—Estrogens (total, in pregnancy) test system.

§862.1275—Estrogens (total, nonpregnancy) test system.

§862.1280—Estrone test system.

§862.1285—Etiocholanolone test system.

§862.1290—Fatty acids test system.

§862.1295—Folic acid test system.

§862.1300—Follicle-stimulating hormone test system.

§862.1305—Formiminoglutamic acid (FIGLU) test system.

§862.1310—Galactose test system.

§862.1315—Galactose-1-phosphate uridyl transferase test system.

§862.1320—Gastric acidity test system.

§862.1325—Gastrin test system.

§862.1330—Globulin test system.

§862.1335—Glucagon test system.

§862.1340—Urinary glucose (nonquantitative) test system.

§862.1345—Glucose test system.

§862.1360—Gamma-glutamyl transpeptidase and isoenzymes test system.

§862.1365—Glutathione test system.

§862.1370—Human growth hormone test system.

§862.1375—Histidine test system.

§862.1377—Urinary homocystine (nonquantitative) test system.

§862.1380—Hydroxybutyric dehydrogenase test system.

§862.1385-17-Hydroxycorticosteroids (17-ketogenic steroids) test system.

§862.1390-5-Hydroxyindole acetic acid/serotonin test system.

§862.1395-17-Hydroxyprogesterone test system.

§862.1400—Hydroxyproline test system.

§862.1405—Immunoreactive insulin test system.

§862.1410—Iron (non-heme) test system.

§862.1415—Iron-binding capacity test system.

§862.1420—Isocitric dehydrogenase test system.

§862.1430-17-Ketosteroids test system.

§862.1435—Ketones (nonquantitative) test system.

§862.1440—Lactate dehydrogenase test system.

§862.1445—Lactate dehydrogenase isoenzymes test system.

§862.1450—Lactic acid test system.

§862.1455—Lecithin/sphingomyelin ratio in amniotic fluid test system.

§862.1460—Leucine aminopeptidase test system.

§862.1465—Lipase test system.

§862.1470—Lipid (total) test system.

§862.1475—Lipoprotein test system.

§862.1485—Luteinizing hormone test system.

§862.1490—Lysozyme (muramidase) test system.

§862.1495—Magnesium test system.

§862.1500—Malic dehydrogenase test system.

§862.1505—Mucopolysaccharides (nonquantitative) test system.

§862.1509—Methylmalonic acid (nonquantitative) test system.

§862.1510—Nitrite (nonquantitative) test system.

§862.1515—Nitrogen (amino-nitrogen) test system.

§862.1520-5′-Nucleotidase test system.

§862.1530—Plasma oncometry test system.

§862.1535—Omithine carbamyl transferase test system.

§862.1540—Osmolality test system.

§862.1542—Oxalate test system.

§862.1545—Parathyroid hormone test system.

§862.1550—Urinary pH (nonquantitative) test system.

§862.1555—Phenylalanine test system.

§862.1560—Urinary phenylketones (nonquantitative) test system.

§862.1565-6-Phosphogluconate dehydrogenase test system.

§862.1570—Phosphohexose isomerase test system.

§862.1575—Phospholipid test system.

§862.1580—Phosphorus (inorganic) test system.

§862.1585—Human placental lactogen test system.

§862.1590—Porphobilinogen test system.

§862.1595—Porphyrins test system.

§862.1600—Potassium test system.

§862.1605—Pregnanediol test system.

§862.1610—Pregnanetriol test system.

§862.1615—Pregnenolone test system.

§862.1620—Progesterone test system.

§862.1625—Prolactin (lactogen) test system.

§862.1630—Protein (fractionation) test system.

§862.1635—Total protein test system.

§862.1640—Protein-bound iodine test system.

§862.1645—Urinary protein or albumin (nonquantitative) test system.

§862.1650—Pyruvate kinase test system.

§862.1655—Pyruvic acid test system.

§862.1660—Quality control material (assayed and unassayed).

§862.1665—Sodium test system.

§862.1670—Sorbitol dehydrogenase test system.

§862.1675—Blood specimen collection device.

§862.1680—Testosterone test system.

§862.1685—Thyroxine-binding globulin test system.

§862.1690—Thyroid stimulating hormone test system.

§862.1695—Free thyroxine test system.

§862.1700—Total thyroxine test system.

§862.1705—Triglyceride test system.

§862.1710—Total triiodothyronine test system.

§862.1715—Triiodothyronine uptake test system.

§862.1720—Triose phosphate isomerase test system.

§862.1725—Trypsin test system.

§862.1730—Free tyrosine test system.

§862.1770—Urea nitrogen test system.

§862.1775—Uric acid test system.

§862.1780—Urinary calculi (stones) test system.

§862.1785—Urinary urobilinogen (nonquantitative) test system.

§862.1790—Uroporphyrin test system.

§862.1795—Vanilmandelic acid test system.

§862.1805—Vitamin A test system.

§862.1810—Vitamin B12 test system.

§862.1815—Vitamin E test system.

§862.1820—Xylose test system.

§862.1825—Vitamin D test system.

Subpart C—Clinical Laboratory Instruments

§862.2050—General purpose laboratory equipment labeled or promoted for a specific medical use.

§862.2100—Calculator/data processing module for clinical use.

§862.2140—Centrifugal chemistry analyzer for clinical use.

§862.2150—Continuous flow sequential multiple chemistry analyzer for clinical use.

§862.2160—Discrete photometric chemistry analyzer for clinical use.

§862.2170—Micro chemistry analyzer for clinical use.

§862.2230—Chromatographic separation material for clinical use.

§862.2250—Gas liquid chromatography system for clinical use.

§862.2260—High pressure liquid chromatography system for clinical use.

§862.2270—Thin-layer chromatography system for clinical use.

§862.2300—Colorimeter, photometer, or spectrophotometer for clinical use.

§862.2310—Clinical sample concentrator.

§862.2320—Beta or gamma counter for clinical use.

§862.2400—Densitometer/scanner (integrating, reflectance, TLC, or radiochromatogram) for clinical use.

§862.2485—Electrophoresis apparatus for clinical use.

§862.2500—Enzyme analyzer for clinical use.

§862.2540—Flame emission photometer for clinical use.

§862.2560—Fluorometer for clinical use.

§862.2680—Microtitrator for clinical use.

§862.2700—Nephelometer for clinical use.

§862.2720—Plasma oncometer for clinical use.

§862.2730—Osmometer for clinical use.

§862.2750—Pipetting and diluting system for clinical use.

§862.2800—Refractometer for clinical use.

§862.2850—Atomic absorption spectrophotometer for clinical use.

§862.2860—Mass spectrometer for clinical use.

§862.2900—Automated urinalysis system.

§862.2920—Plasma viscometer for clinical use.

Subpart D—Clinical Toxicology Test Systems

§862.3030—Acetaminophen test system.

§862.3035—Amikacin test system.

§862.3040—Alcohol test system.

§862.3050—Breath-alcohol test system.

§862.3100—Amphetamine test system.

§862.3110—Antimony test system.

§862.3120—Arsenic test system.

§862.3150—Barbiturate test system.

§862.3170—Benzodiazepine test system.

§862.3200—Clinical toxicology calibrator.

§862.3220—Carbon monoxide test system.

§862.3240—Cholinesterase test system.

§862.3250—Cocaine and cocaine metabolite test system.

§862.3270—Codeine test system.

§862.3280—Clinical toxicology control material.

§862.3300—Digitoxin test system.

§862.3320—Digoxin test system.

§862.3350—Diphenylhydantoin test system.

§862.3380—Ethosuximide test system.

§862.3450—Gentamicin test system.

§862.3520—Kanamycin test system.

§862.3550—Lead test system.

§862.3555—Lidocaine test system.

§862.3560—Lithium test system.

§862.3580—Lysergic acid diethylamide (LSD) test system.

§862.3600—Mercury test system.

§862.3610—Methamphetamine test system.

§862.3620—Methadone test system.

§862.3630—Methaqualone test system.

§862.3640—Morphine test system.

§862.3645—Neuroleptic drugs radioreceptor assay test system.

§862.3650—Opiate test system.

§862.3660—Phenobarbital test system.

§862.3670—Phenothiazine test system.

§862.3680—Primidone test system.

§862.3700—Propoxyphene test system.

§862.3750—Quinine test system.

§862.3830—Salicylate test system.

§862.3850—Sulfonamide test system.

§862.3870—Cannabinoid test system.

§862.3880—Theophylline test system.

§862.3900—Tobramycin test system.

§862.3910—Tricyclic antidepressant drugs test system.

§862.3950—Vancomycin test system.

What is claimed:

1. A lancet suited for use in drawing blood or other bodily fluid, wherein the lancet is provided with a region where a chemical reagent resides, the chemical reagent including one or more agents that react with at least one constituent of the bodily fluid to ascertain information about the bodily fluid from which it was drawn, the lancet has a body and a head that is provided with a relatively sharp tip for penetrating the skin of the person, the tip is provided with at least one channel or groove in fluid communication with the area where the chemical reagent is positioned, the channel or groove transports the fluid from the tip to the area where the chemical reagent is positioned, the channels extend between the tip of the lancet and the recess, an electroconductive ink is positioned on the lancet wherein the ink is patterned into electrical contacts, part of which extend into the area where the chemical reagent is deposited and a part of which extend to a location away from the area where the chemical reagent is deposited, the chemical reagent positioned on the lancet undergoes a reaction with the bodily fluid that creates an electrical potential between the two ends of the electrical contacts.

2. The lancet of claim 1 wherein the lancet has a pencil-like shape, with the head resembling the conically-shaped pencil point and the body resembling a cylindrical barrel.

3. The lancet of claim 2 wherein the lancet is integrated into a diagnostic test kit in which the lancet is driven out of the recess to penetrate a person's skin, draw a sample of a fluid, which by capillary action is transported through the grooves to the area where the reagent is positioned, the lancet is returned to within the protective recess, and wherein a chemical reaction occurs, generating an electrical potential, which is measured by the diagnostic test kit, which then converts the value of the electrical potential.

4. A test strip for use in analyzing a sample drawn from a person, wherein the test strip has a substrate, a through hole where a chemical reagent can be deposited and cured, electrical contacts that extend from where the reagent is positioned to another area on the strip, wherein a wicking region comprises grooves containing a non-compatible ink, the wicking region transports bodily fluid from the end of the strip to the region where the chemical reagent is deposited, the strip has an overlayer of a carrier material.
